(19) United States
(12) Patent Application Publication
(10) Pub. No.: US 2007/0219225 A1
(43) Pub. Date: Sep. 20, 2007

(54) Pharmaceutical Compositions For The Prevention And Treatment Of Atherosclerosis

(75) Inventors: Remy Couderc, Bourg La Reine (FR); Carole Rasmusen, Beauchamp (FR); Chantal Martin, Paris (FR); Viviane Tricottet, Dammarie Les Lys (FR); Luc Cynober, Sceaux (FR); Christophe Moinard, Bourg La Reine (FR)

Correspondence Address: YOUNG & THOMPSON, 745 SOUTH 23RD STREET, 2ND FLOOR, ARLINGTON, VA, 22202, US

(73) Assignee: UNIVERSITE RENE DESCARTES-PARIS 5, Paris Cedex 06 (FR)

(21) Appl. No.: 11/587,426

(22) PCT Filed: Apr. 29, 2005
(86) PCT No.: PCT/FR05/01083

§ 371 Date: Jan. 26, 2007

(30) Foreign Application Priority Data
Apr. 29, 2004 (FR) 0404569

(51) Int. Cl. A61K 31/40; A61K 31/215; A61K 31/403; A61K 31/505; A61K 31/351

(52) U.S. Cl. 514/275; 514/419; 514/460; 514/547

(57) ABSTRACT

The present invention relates to the use, of at least one statin, or of a pharmaceutically acceptable salt thereof, in combination with
at least one compound of the following formula (I): 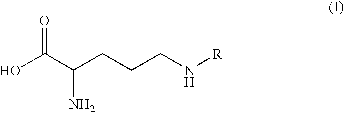
in which R represents, —H, 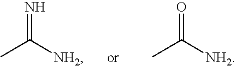 or a pharmaceutically acceptable salt thereof, for the preparation of a medicament intended for the prevention or treatment of atherosclerosis, in particular of primary or secondary atherosclerosis, hypertension, diabetes, or neurodegenerative diseases, in particular Alzheimer's disease.

The present invention relates to a novel pharmaceutical composition and its use in particular within the framework of combating atherosclerosis.

Atherosclerosis is the first cause of mortality in the industrialized countries, with more than 20% of deaths (Murray J L, Lopez A D. Mortality by cause for eight regions of the world: global burden of disease study. Lancet 1997; 349: 1269-76). According to the WHO (1954), atherosclerosis is a variable combination of changes to the intima of the large and medium-calibre arteries involving the formation of an atheromatous plaque, i.e. a local accumulation of lipids, complex carbohydrates, blood and blood products, fibrous tissue and calcareous deposits; all accompanied by modifications of the media. This pathology manifests itself only after several years of sub-clinical evolution. Its prevention, even more than its treatment, therefore remains of the utmost importance.

The formation of the plaque is a succession of five stages (Tedgui A, Mallat Z. Formation de la plaque d'athérosclérose. Rev Prat 1999; 49: 2081-2086):
LDL (Low Density Lipoprotein) accumulation in the intima,
LDL oxidation (LDLox) in the wall by contact with endothelial cells, smooth muscle cells (SMCs) or macrophages,
recruitment of circulating monocytes by means of the induction of adhesion molecules, such as VCAM1 or ICAM1, caused by oxidized LDLs,
LDLox trapping by the macrophages by means of the scavenger receptors; thus cholesterol accumulates in the macrophages,
formation of a stabilizing fibromuscular cap, constituted by smooth muscle cells and proteins from the extracellular matrix.

L-Arginine, a basic amino acid present in plant and animal proteins, is essential to the synthesis of tissue proteins. In different animal studies, when the diet is supplemented with L-Arginine (2% in the drinking water, a dose making it possible to double the plasmatic arginine concentration), it seems that the latter tends to have an antiatherogenic effect. This treatment also seems to have an effect on vascular reactivity. Böger et al. (Böger R H, Bode-Böger S M, Brandes R P, Phivthong-ngam L, Böhme M, Nafe R, et al. Dietary L-Arginine reduces the progression of atherosclerosis in cholesterol-fed rabbits. Circulation 1997; 96: 1282-1290), as well as Behr-Roussel et al. (Behr-Roussel D, Rupin A, Simonet S, Bonhomme E, Coumailleau S, Cordi A, et al. Effect of chronic treatment with the inducible nitric oxide synthase inhibitor N-iminoethyl-L-lysine or with L-arginine on progression of coronary and aortic atherosclerosis in hypercholesterolemic rabbits. Circulation 2000; 102: 1033-1038) demonstrate a stabilization of the atheromatous plaque area with an enriched diet over 12 to 16 weeks.

Statins are powerful cholesterol synthesis inhibitors. Their main indication is the reduction of plasmatic LDL-cholesterol in primary or secondary prevention of cardiac ischemic accidents (Vaughan C J, Murphy M B, Buckley B M. Statins do more than just lower cholesterol. Lancet 1996; 348: 1079-1082).

Statins inhibit HMG-CoA reductase by reversible competition with the substrate HMG-CoA, for the active site of the enzyme. They therefore lead to a reduction in the synthesis of mevalonate and its derivatives including cholesterol. Therefore, statins are indicated for the treatment of atherosclerosis.

However, at present, none of the treatments mentioned makes it possible to completely prevent and/or treat atherosclerosis.

An objective of the invention is therefore to provide a more effective method for the prevention and/or treatment of atherosclerosis.

The present invention follows in particular from the demonstration of an unexpected synergistic effect of the combination of a statin and L-Arginine within the framework of the prevention and/or treatment of atherosclerosis.

Thus, the present invention relates to the use,
of at least one statin, or of a pharmaceutically acceptable salt thereof, in combination with
at least one compound of the following formula (I): 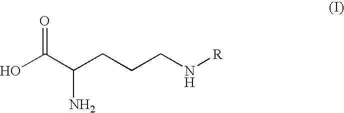
   in which R represents, —H, 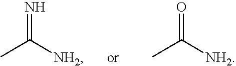
   or a pharmaceutically acceptable salt thereof, for the preparation of a medicament intended for the prevention or treatment of atherosclerosis, in particular of primary or secondary atherosclerosis, hypertension, diabetes, or neurodegenerative diseases, in particular Alzheimer's disease.
   When R represents —H, the compound of formula (I) corresponds to ornithine.
   When R represents 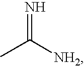
   the compound of formula (I) corresponds to arginine.
   When R represents 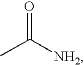
   the compound of formula (I) corresponds to citrulline.

These three amino acids possess a common metabolism in the organism.

Hypertension, diabetes and the neurodegenerative diseases, in particular Alzheimer's disease, have a vascular component linked to an endothelial dysfonction which can be compensated for by the synergistic combination of a compound of formula (I) and statin, in particular by the arginine-statin synergistic combination, according to the invention.

The expression “in combination” means that the statin and the compound of formula (I) are both present independently in the medicaments according to the invention, where they are in no event interlinked, either by means of weak bonds, such as electrostatic interactions, in order to form a salt for example, or by means of strong bonds, such as covalent bonds.

As is meant here, the word “statin” designates a compound belonging to the class of HMG-CoA reductase inhibitors. In particular, the term statin in no event corresponds to an amino acid derivative originating from pepstatin and having renin-inhibiting properties.

Moreover, advantageously, the compound of general formula (I), in particular arginine, is used as an adjuvant in order to increase the effects of the statins within the framework of the preparation of medicaments intended for the prevention or treatment of atherosclerosis, in particular of primary or secondary atherosclerosis, hypertension, diabetes, or neurodegenerative diseases, in particular Alzheimer's disease.

According to a preferred embodiment of the invention, the statin is chosen from the group comprising:
atorvastatin, pravastatin, fluvastatin, rosuvastatin, lovastatin and simvastatin.

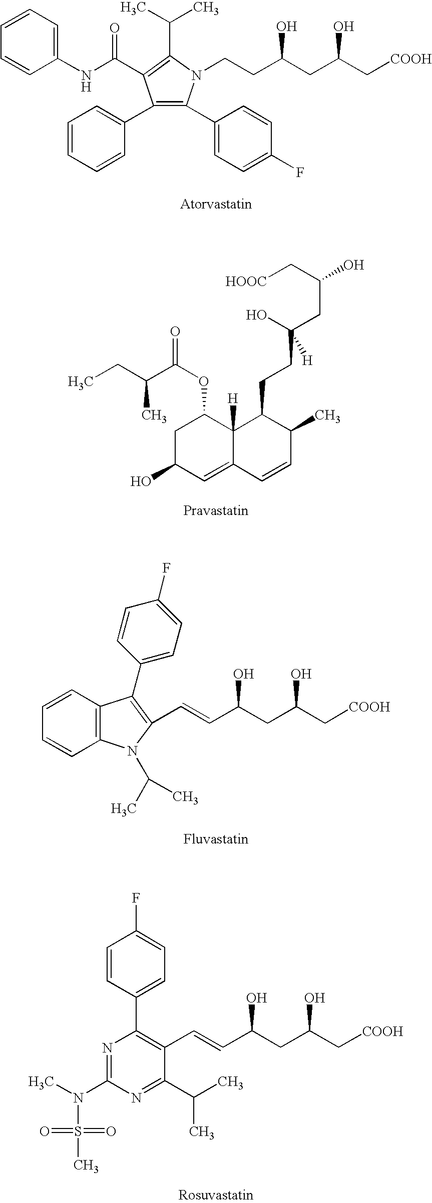

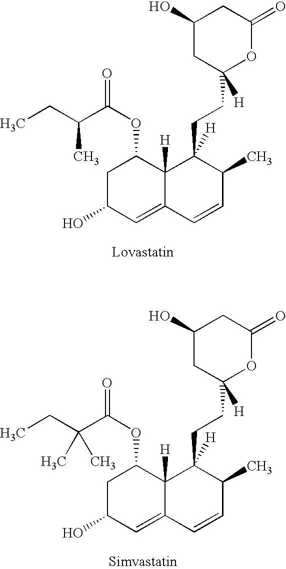

This list is not limitative, all the other statins known to a person skilled in the art can also be used according to the invention.

According to a particularly preferred embodiment of the invention, the statin is atorvastatin.

According to another preferred embodiment of the invention, the compound of formula (I) corresponds to the L-isomer.

According to yet another preferred embodiment of the invention, R represents 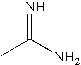
the compound of formula (I) then corresponding to arginine, in particular L-Arginine.

The invention relates more particularly to the use as defined above, of a statin, in particular atorvastatin, in a quantity suitable for the administration to an individual of a dose from about 5 mg/day to about 80 mg/day.

The invention also relates more particularly to the use as defined above, of a compound of formula. (I), in particular L-Arginine, in a quantity suitable for the administration to an individual of a dose from about 1 g/day to about 30 g/day, in particular from about 5 g/day to about 30 g/day.

Advantageously doses of L-Arginine of less than 5 g/day are particularly suitable for children.

The invention also relates quite particularly to the use, as defined above, of L-Arginine, in a quantity suitable for the administration to an individual of a dose of at least about 10 g/day.

In a particular embodiment, the invention relates to the use, as defined above, of L-Arginine in a quantity suitable for the administration to an individual of a dose of about 0.15 g/kg/day, i.e. a dose of 0.15 g/day of L-Arginine per kg of bodyweight of the individual in question.

According to another particular embodiment of the use as defined above, the medicament is suitable for the administration to an individual of a single dose from about 5 mg to about 80 mg of statin and from about 5 g to about 30 g, in particular about 10 g, of L-Arginine.

The present invention also relates to products containing
at least one statin, or a pharmaceutically acceptable salt thereof, and
at least one compound of the following formula (I): 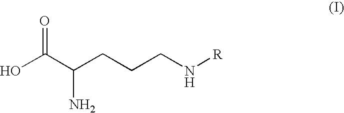
   in which R represents, —H, 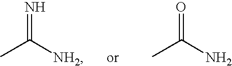
   or a pharmaceutically acceptable salt thereof,
      as combination products for simultaneous or separate use or use spread over time within the framework of the treatment of atherosclerosis, in particular of primary or secondary atherosclerosis, hypertension, diabetes, or neurodegenerative diseases, in particular Alzheimer's disease.

The expression “combination products” signifies that the statin and the compound of formula (I) are both present independently in the products according to the invention, where they are in no event interlinked, either by means of weak bonds, such as electrostatic interactions, in order to form a salt for example, or by means of strong bonds, such as covalent bonds.

According to a preferred embodiment of the invention, the statin is chosen from the group comprising:

atorvastatin, pravastatin, fluvastatin, rosuvastatin, lovastatin and simvastatin.

According to a particularly preferred embodiment, the statin is atorvastatin.

According to another preferred embodiment of the invention, the compound of formula (I) corresponds to the L-isomer.

According to yet another preferred embodiment of the invention, R represents 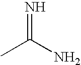
the compound of formula (I) then corresponding to arginine, in particular L-Arginine.

According to a particularly preferred embodiment of the invention, said products are suitable for the administration to an individual of a dose from about 5 mg/day to about 80 mg/day of statin, in particular atorvastatin.

According to another particularly preferred embodiment of the invention, said products are suitable for the administration to an individual of a dose from about 1 g/day to about 30 g/day, in particular from about 5 g/day to about 30 g/day, of a compound of formula (I), in particular L-Arginine.

Advantageously doses of L-Arginine of less than 5 g/day are particularly suitable for children.

According to another particularly preferred embodiment of the invention, said products are suitable for the administration to an individual of a dose of L-Arginine of at least about 10 g/day.

In a particular embodiment, said products are suitable for the administration to an individual of a dose of L-Arginine of about 0.15 g/kg/day.

According to another particular embodiment of the products as defined above, the products are suitable for the administration to an individual of a single dose from about 5 mg to about 80 mg of statin and about 5 g to about 30 g, in particular about 10 g, of L-Arginine.

The present invention also relates to a pharmaceutical composition comprising as active ingredient:
at least one statin, or a pharmaceutically acceptable salt thereof, and
at least one compound of the following formula (I): 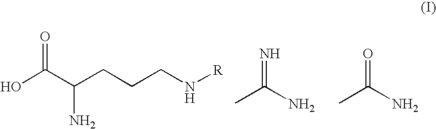
in which R represents, —H, or
or a pharmaceutically acceptable salt thereof,
   in combination with a pharmaceutically acceptable vehicle.
   When R represents —H, the compound of formula (I) corresponds to ornithine.
   When R represents 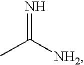
   the compound of formula (I) corresponds to arginine.
   When R represents 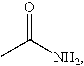
   the compound of formula (I) corresponds to citrulline.
   These three amino acids possess a common metabolism in the organism.

As is meant here, the statin and the compound of formula (I) are both present independently in the pharmaceutical compositions according to the invention, where they are in no event interlinked, either by means of weak bonds, such as electrostatic interactions, in order to form a salt for example, or by means of strong bonds, such as covalent bonds.

According to a preferred embodiment of the invention, the statin is chosen from the group comprising:

atorvastatin, pravastatin, fluvastatin, rosuvastatin, lovastatin and simvastatin.

This list is not limitative, all the other statins known to a person skilled in the art can also be used according to the invention.

According to a particularly preferred embodiment of the invention, the statin is atorvastatin.

According to another preferred embodiment of the invention, the compound of formula (I) corresponds to the L-isomer.

The natural amino acids found in the organism are essentially of L type.

According to yet another preferred embodiment of the invention, R represents 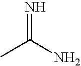
the compound of formula (I) then corresponding to arginine, in particular to L-Arginine.

According to another preferred embodiment of the invention, the pharmaceutical composition according to the invention is suitable for the administration to an individual of a dose from about 5 mg/day to about 80 mg/day of statin, in particular atorvastatin.

According to yet another preferred embodiment of the invention, the pharmaceutical composition according to the invention is suitable for the administration to an individual of a dose from about 1 g/day to about 30 g/day, in particular from about 5 g/day to about 30 g/day, of a compound of formula (I), in particular L-Arginine.

Advantageously doses of L-Arginine of less than 5 g/day are particularly suitable for children.

According to a particularly preferred embodiment, the pharmaceutical composition according to the invention is suitable for the administration to an individual of a dose of L-Arginine of at least about 10 g/day.

In a particular embodiment, the pharmaceutical composition as defined above is suitable for the administration to an individual of a dose of L-Arginine from about 0.15 g/kg/day.

According to another particular embodiment of the pharmaceutical compositions as defined above, the pharmaceutical compositions are suitable for the administration to an individual of a single dose from about 5 mg to about 80 mg of statin and from about 5 g to about 30 g, in particular about 10 g, of L-Arginine.

According to another preferred embodiment of the invention, the pharmaceutical composition according to the invention is suitable for administration by oral route.

According to yet another preferred embodiment of the invention, the pharmaceutical composition according to the invention is characterized in that it is presented in the form of a powder to be diluted, pills, sachets, tablets, capsules, or any other acceptable galenic form.

### DESCRIPTION OF THE FIGURES

FIG. 1

FIG. 1 represents the development of the weight of the animals (in kilograms, y-axis) as a function of time (x-axis) from their arrival, then from the start (T0) to the end of the treatment (weeks T1 to T8) for the control group (first histogram), the arginine group (second histogram), the statin group (third histogram) and the statin+arginine group (fourth histogram).

FIG. 2

FIG. 2 represents the animals' average food intake (in grams/day, y-axis) as a function of time (x-axis) from the start of the treatment (T0) to the end of the treatment (weeks T1 to T8) for the control group (first histogram), the arginine group (second histogram), the statin group (third histogram) and the statin+arginine group (fourth histogram).

FIG. 3

FIG. 3 represents the average cholesterolaemia (in mmol/l, y-axis) for the control group, the arginine group, the statin group and the statin+arginine group, at times (x-axis) T0 (first histogram), T4 (second histogram), T8 (sacrifice) (third histogram).

FIG. 4

FIG. 4 represents the average triglyceridaemia (in mmol/l, y-axis) for the control group, the arginine group, the statin group and the statin+arginine group, at times (x-axis) T0 (first histogram), T4 (second histogram), T8 (sacrifice) (third histogram).

FIG. 5

FIG. 5 represents the average plasmatic HDL concentration (in mmol/l, y-axis) for the control group, the arginine group, the statin group and the statin+arginine group, at times (x-axis) T0 (first histogram), T4 (second histogram), T8 (sacrifice) (third histogram).

FIG. 6

FIG. 6 represents the average plasmatic LDL concentration calculated according to the Friedewald formula (in mmol/l, y-axis) for the control group, the arginine group, the statin group and the statin+arginine group, at times (x-axis) T0 (first histogram), T4 (second histogram), T8 (sacrifice) (third histogram).

FIG. 7

FIG. 7 represents the average plasmatic arginine concentration (in μmol/l, y-axis) for the control group, the arginine group, the statin group and the statin+arginine group, at times (x-axis) T0 (first histogram), T4 (second histogram), T8 (sacrifice) (third histogram).

FIG. 8

FIG. 8 represents the average plasmatic ornithine concentration (in μmol/l, y-axis) for the control group, the arginine group, the statin group and the statin+arginine group, at times (x-axis) T0 (first histogram), T4 (second histogram), T8 (sacrifice) (third histogram).

FIG. 9

FIG. 9 represents the plasmatic citrulline concentration (in μmol/l, y-axis) for the control group, the arginine group, the statin group and the statin+arginine group, at times (x-axis) T0 (first histogram), T4 (second histogram), T8 (sacrifice) (third histogram).

FIG. 10

FIG. 10 represents the percentage of the surface area of the lesions (y-axis) in the different groups (x-axis) at the time of sacrifice (T8).

FIG. 11

FIG. 11 represents the percentage of the surface area of the lesions (y-axis) at the distal level of the aorta in the different groups (x-axis) at the time of sacrifice (T8).

### EXAMPLE

### Materials and Methods

1. Animals and Experimental Protocol

Twenty-four Watanabe rabbits (Centre de Production Animale, Olivet, France) which are hyperlipidaemic due to an hereditary LDL-receptor (LDL-R) deficiency, homozygous, and six weeks old, are used. Each animal is placed in an individual cage and receives a food ration of 200 g to 250 g per day. After acclimatization to the animal house for 7 days, four groups of six rabbits are formed:
a control group receives a normal rabbit diet containing 16% proteins, 3.2% lipids and 49.3% carbohydrates (EXT C15, Dietex, St Gratien, France),
the rabbits in the second group, the so-called “arginine” group, receive a diet enriched with L-Arginine so as to double their plasmatic concentration of this amino acid. L-Arginine (Sigma, St Quentin Fallavier, France) is mixed with the food, at a rate of 15 g/kg of food producing an intake of 1 g of arginine/kg of live weight/day,
a third group, the so-called “statin” group, receives a diet with a statin (Atorvastatin, Tahor® 10 mg, Godeche-Pfizer, Freiburg, Germany) in the drinking water, at a rate of 2.5 mg/kg/day (Maestro R, Aragoncillo P et al. Effect of Atorvastatin on endothelium-dependant constrictor factors in dyslipidemic rabbits. Gen Pharma. 2000; 34: 263-272),
and finally a fourth group, the so-called “statin/arginine group”, receives a diet enriched with arginine and treatment with atorvastatin, also at 2.5 mg/kg/day.

The intake of L-Arginine in the enriched diet is such as to correspond to an L-Arginine intake of about 0.15 g/kg/day in humans.

The food intake is measured daily and the animals are weighed once a week. Plasma samples are taken every fifteen days from the marginal ear vein. The blood, collected in heparin tubes, is centrifuged (4° C., 2000 g, 15 minutes). Part of the plasma is deproteinized by 30 a solution of sulphosalicylic acid (30%) then divided into aliquot fractions with a view to an assay of the amino acids. The remainder of the plasma is aliquoted into several fractions of about 200 μl, and stored at −80° C.

After eight weeks, a sampling of serum (about 1 ml) is carried out then the animals are anaesthetized with an injection of heparinized pentobarbital, and finally sacrificed.

The whole study is carried out in accordance with current regulations relating to animal experimentation.

2. Biochemical Analyses

The lipid profile, i.e., cholesterol, triglycerides, HDL (High Density Lipoprotein) and LDL (Low Density Lipoprotein) cholesterol, is determined on a Hitachi 911 by the usual methods. The cholesterol is assayed according to an enzymatic colorimetric test using a cholesterol-esterase and a cholesterol-oxidase, followed by a reaction with a peroxidase. The latter reacts with hydrogen peroxide and releases a red derivative the stain intensity of which (measured between 550 and 700 nm) is directly proportional to the cholesterol concentration. The triglyceride assay is also based on this principle, using a lipase which releases glycerol, and a peroxidase which reacts with the hydrogen peroxide and forms a red compound. The HDL cholesterol is assayed by an enzymatic colorimetric test in two phases. The first selects the different lipid fractions by means of dextran sulphate. The second stage is similar to the cholesterol assay, involving a cholesterol-esterase and a cholesterol-oxidase modified by polyethylene glycol, and a peroxidase which releases a blue-violet derivative. The results are expressed in mmol/l. The kits necessary for all these assays are supplied by RANDOX (Montpellier, France). The LDLs are calculated using the Friedewald formula, from the total cholesterol, the HDL cholesterol, and the triglycerides (Friedewald et al. (1972) Clin. Chem. 18: 499-502).

The plasmatic arginine, ornithine and citrulline concentrations are determined on a JEOL (Tokyo, Japan) amino acid analyzer. The amino acids are separated by cation-exchange chromatography. On leaving the column, they are developed by a ninhydrin stain reaction. The reaction obtained is quantified by photometry at 570 and 440 nm. The results are expressed in μmol/l.

3. Morphological and Histological Study

On the day of sacrifice, after anaesthesia, in the shortest possible time, the animal is opened along the whole length of the cervico-thoracic region. The aorta is incised as low as possible. Using curved scissors, the aorta is removed from the aortic arch as far as the level of the iliac bifurcation following the vertebral column. The aorta is first cleaned in physiological serum and the fatty tissues are removed from its external tunica. It is fixed for 20 minutes in a 2% paraformaldehyde−2% glutaraldehyde mixture then opened along its length. It is then again immersed in the same fixer for 24 hours, after which the aorta is rinsed in 70% ethanol, then stained with Soudan IV for 15 minutes. It is once again rinsed in 80% ethanol for 20 minutes then under running water for 1 hour (22). Then the aorta is photographed flat with a Nikon 995 digital camera. The images are analyzed by means of planimetry software (Scion Image) which makes it possible to determine the surface of the lesions. Finally the aorta is fixed in the same fixer with a view to microscopic study.

For optical microscopy, the aorta is cut into four segments:
one at the start and one at the end of the aortic arch,
one in the middle and one at the end of the aorta.

Adjacent fragments are sampled for the electron microscopy.

The samples are progressively dehydrated in ethanol up to 100% ethanol then treated with paraffin. The sections thus impregnated with paraffin are included according to an oriented inclusion which makes it possible to have transversal sections. 5 μm sections are produced, which are stuck onto slides then stained with HES (haemalum/erythrosine/saffron).

4. Statistical Analyses

StatView software, version 5.0, was used for the statistical analysis of the data. All the values are given as the average—SEM. Comparison of the groups is carried out by a Kruskal-Wallis test. This test is a non-parametric version of variance analysis with a ranking factor. Comparison between two groups is carried out by a Mann-Whitney test, which is the non-parametric version of the t-test on independent series. Significant values for p≦0.05 are considered. The significance of the correlations between quantitative variables is assessed by the Spearman test.

### RESULTS

The treatment was carried out over 8 weeks (from TO at the start of the treatment to T8).

1. Animals and General Parameters

1.1. Monitoring of the Animals: Weight and Food Intake

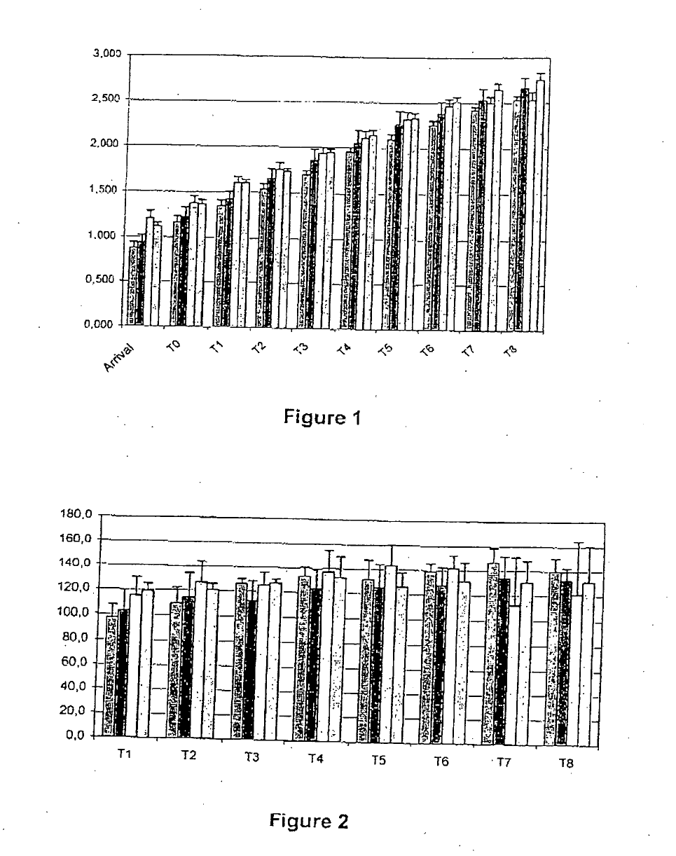

During the experimentation, the animals in the four groups have an identical growth curve (FIG. 1). On arrival at the animal house, the animals in the control group and the arginine group are smaller, and this difference from the statin group and the statin/arginine group becomes less distinct from the start of the treatment (T0).

The average food intake (FIG. 2) increases between the period T0-T4, then tends to be regular up to T8. The reduction in the food intake of the statin group after T5 is linked to the presence of two sick animals. Moreover, two animals in the arginine group died before the end of the protocol and have not been included in the study.

The measurement of the weights and daily food intake reflects the homogeneity of the animals with respect to these parameters whatever the group. At age 10 weeks (T4), their weight is comprised between 1.9 and 2.1 kg which corresponds to the data found in the literature (Murakami S, Kondo Y, Sakurai T, Kitajima H, Nagate T. Taurine suppresses development of atherosclerosis in Watanabe heritable hyperlipidemic (WHHL) rabbits. Atherosclerosis. 2002; 163: 79-87). The food intake increases as a function of age (from 97.6 to 139.5 g/day for the control group for example) and remains relatively constant after T3.

1.2. The Lipid Profile

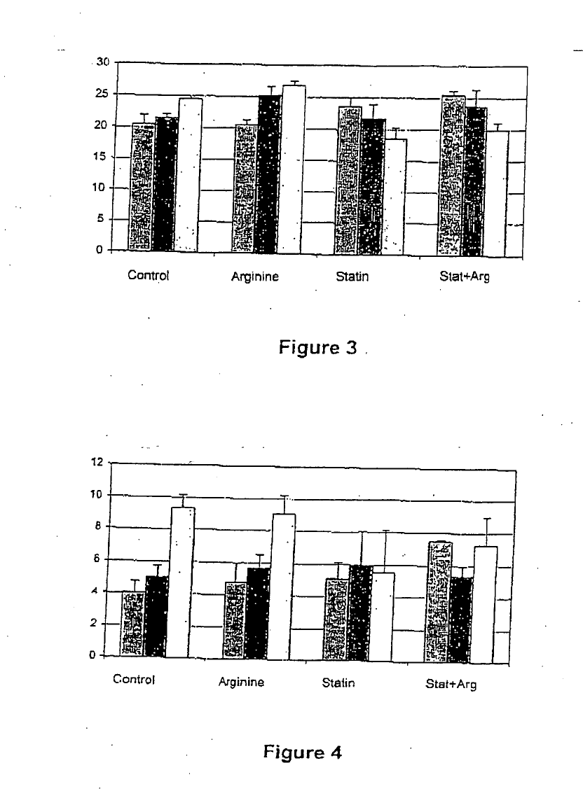

Treatments with atorvastatin and with atorvastatin plus arginine have a beneficial effect on the plasmatic cholesterol (FIG. 3); The latter diminishes during treatment. This reduction is significant for the two groups versus the control at time T8. The arginine diet has no effect on the cholesterol.

The plasmatic triglycerides concentration (FIG. 4) increases with age in the control group and the arginine group. A significant positive effect is noted in animals treated with atorvastatin alone (versus the control) at T8: the triglycerides concentration does not increase. This effect is also observed for the statin/arginine group (not significant).

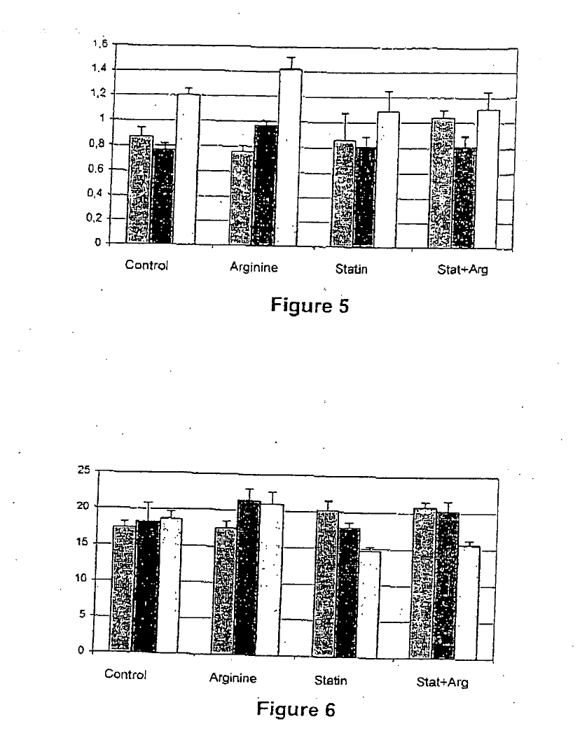

The HDL concentration (FIG. 5) in the different groups develops in the same way as the triglycerides during the protocol.

Similarly, the LDL concentration (FIG. 6) follows the same line as that of the cholesterol. The treatment with atorvastatin induces a reduction in this concentration, similarly for the atorvastatin plus arginine treatment.

On the one hand, the lipid profile is consistent with the literature: our animals at the start of the study (six weeks old) have cholesterolaemia between 20.4 mmol/l (control group) and 25.4 mmol/l (statin/arginine group), consistent with the work of Clubb et al. (Clubb F J, Cerny J L, Deferrari D A, Butler-Aucoin M M, Willerson J T, Buja L M. Development of atherosclerotic plaque with endothelial disruption in Watanabe heritable hyperlipidemic rabbit aortas. Cardiovasc Pathol 2001; 10: 1-11) which reports an average value of 21.9±1.5 mmol/l. On the other hand, Dowell et al. (Dowell F J, Hamilton C A, Lindop G B, Reid J L. Development and progression of atherosclerosis in aorta from heterozygous and homozygous WHHL rabbits. Effects of simvastatin treatment. Arterioscler Thromb Vasc Biol. 1995; 15: 1152-60) have carried out a comparative study between heterozygous and homozygous Watanabe rabbits and have defined the homozygous rabbits as having a cholesterol level of more than 10 mmol/l, which is indeed the case with our animals.

Between T0 and T8, in the control group and the arginine group an increase in the plasmatic cholesterol concentration of 20% and 30% respectively is observed with age (FIG. 3) whereas with the statin and statin/arginine treatment this concentration is reduced by 27% for the two groups. Thus according to our results, atorvastatin has a cholesterol- lowering effect in homozygous animals: it could act at the level of hepatic synthesis of cholesterol by reducing the latter. Similar results are found in the literature but with different models: a study with lovastatin, spread over 16 weeks involving the New Zealand White rabbit (Böger R H, Bode-Böger S M, Brandes R P, Phivthong-ngam L, Böhme M, Nafe R, et al. Dietary L-Arginine reduces the progression of atherosclerosis in cholesterol-fed rabbits. Circulation 1997; 96: 1282-1290) shows a 32% reduction in cholesterol. Moreover, a recent study (Suzuki H, Kobayashi H, Sato F, Yonemitsu Y, Nakashima Y, Sueishi K. Plaque-Stabilizing Effect of Pitavastatin in Watanabe Heritable Hyperlipidemic (WHHL) Rabbits. J Atheroscler Thromb. 2003; 10: 109-16) with Watanabe rabbits over 16 weeks, and using pitavastatin, shows a 28.6% reduction in plasmatic cholesterol. The reduction in cholesterolaemia relates mainly to the LDL fraction as shown by the results presented (27.16% reduction for the statin group and 24.5% for the statin/arginine group), data also found in the study by Suzuki et al.

As regards the triglycerides, the same profile is observed for the control group and the arginine group: the triglycerides increase by more than half during the protocol (between T0 and T8). By contrast, the animals treated with statin (statin group and statin/arginine group) have a virtually constant concentration between T0 and T8, which implies a positive effect of the atorvastatin against hypertriglyceridaemia.

Arginine alone appears to have no appreciable effect on this lipid profile, which is consistent with numerous works (Böger R H, Bode-Böger S M, Brandes R P, Phivthong-ngam L, Böhme M, Nafe R, et al. Dietary L-Arginine reduces the progression of atherosclerosis in cholesterol-fed rabbits. Circulation 1997; 96: 1282-1290; Brandes R P, Brandes S, Böger R H, Bode-Böger S M, Mugge A. L-Arginine supplementation in hypercholesterolemic rabbits normalizes meukocyte adhesion to non-endothelial matrix. Life Sci 2000; 66: 1519-1524; Singer A H, Tsao P S, Wang B Y, Bloch D A, Cooke J P. Discordant effects of dietary L-arginine on vascular structure and reactivity in hypercholesterolemic rabbits. J Cardiovasc Pharmacol 1995; 25: 710-716).

1.3. Concentration of Arginine, Ornithine, and Citrulline

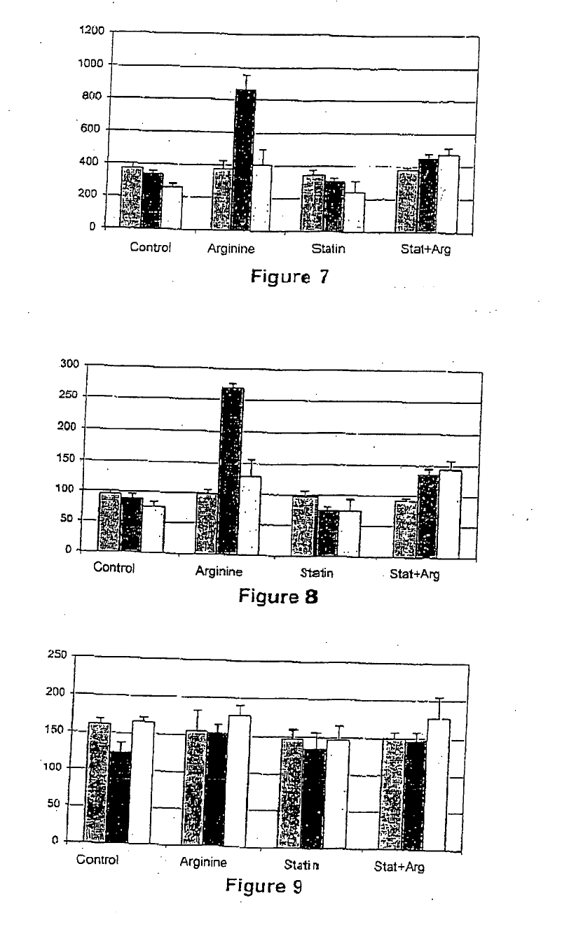

The plasmatic arginine concentration (FIG. 7) is the same for the control group and the statin group. The latter is doubled in the arginine group at T4 (significant versus the control), but drops at T8. The increase in the arginine concentration in the statin/arginine group is regular but remains much lower than that observed for the arginine group.

The ornithine profile (FIG. 8) is similar to that of the arginine.

The plasmatic citrulline concentration (FIG. 9) for the statin/arginine group more or less follows the arginine concentration.

In general, the L-Arginine concentration (this parameter was assayed at times T0, T2, T4, T6 and T8, data not shown) doubled in the treated groups, i.e. the arginine group and the statin/arginine group.

3. Morphological and Histological Study of Arterial Lesions

3.1. Macroscopic Study of the Aortas

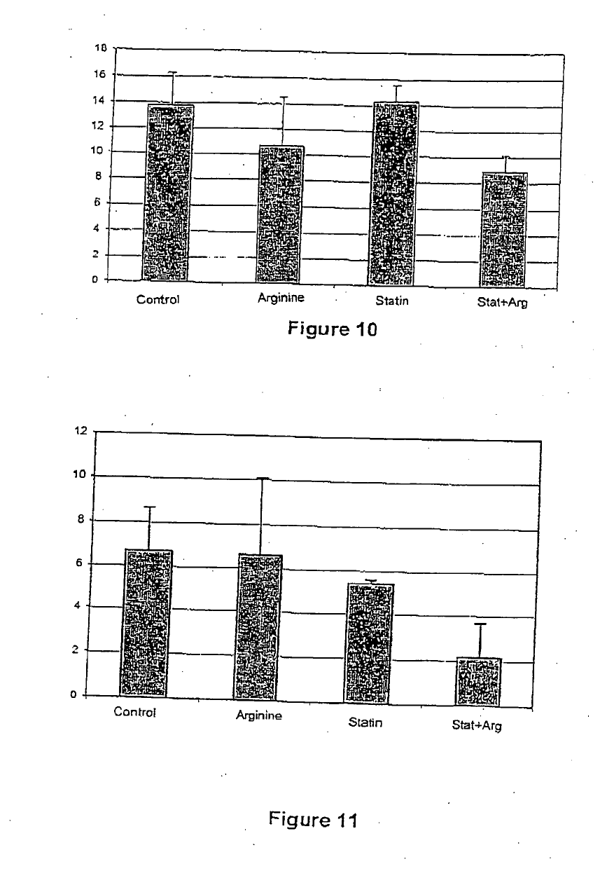

Evaluation of the surface area of the lesions (FIG. 10) by planimetry shows that the latter is smaller with arginine alone, and also significantly smaller with arginine/statin (versus the control). On the other hand, no difference is observed between the control group and the group treated with statin alone. Surprisingly, the statin/arginine combination therefore exhibits a synergistic effect on the reduction of the surface of the lesions.

If interest is confined to the distal half of the aorta, the results are very significant (FIG. 11): with atorvastatin/arginine treatment, the surface area of the lesions is significantly reduced. The combination of the administration of atorvastatin and arginine exhibits unexpected synergistic effects on the reduction of the atherosclerotic lesions because the treatments with atorvastatin alone and arginine alone have no effect.

3.2. Microscopic Study of the Arterial Wall

Observation of the slides shows a smaller thickness of the lesions for the statin/arginine group compared with the statin group. The larger lesions are found in the two firsts sections (at the start and end of the aortic arch); on the last two sections (middle and end of the aorta), the lesions are less frequent. As regards their composition, the majority of lesions are more fibrous than cellular (comprising spumous and some polynuclear macrophages). There is no particular tendency as regards the composition of the lesions between the groups.

At macroscopic level, the lesion surface is unchanged in the statin group versus the control group, whereas numerous works are of the opposite opinion, in particular that of Kroon et al. (Aliev G, Burnstock G. Watanabe rabbits with heritable hypercholesterolaemia: a model of atherosclerosis. Histol Histopathol. 1998; 13: 797-817). After administration of pravastatin at a rate of 40 mg/kg/day for nine months, it shows a 53 to 80% reduction in the incidence of lesions. Similarly, Maeso et al. show a reduction in the size of the lesions in New Zealand White rabbits treated with atorvastatin, 2.5 mg/kg/day (Maeso R, Aragoncillo P et al. Effect of Atorvastatin on endothelium-dependant constrictor factors in dyslipidemic rabbits. Gen Pharma. 2000; 34: 263-272). Also, Böger (Böger R H, Bode-Böger S M, Brandes R P, Phivthong-ngam L, Böhme M, Nafe R, et al. Dietary L-Arginine reduces the progression of atherosclerosis in cholesterol-fed rabbits. Circulation 1997; 96: 1282-1290) has shown that lovastatin slowed down the plaque progression in New Zealand White rabbits.

Moreover, the arginine group has a lesion surface which is smaller than the control by a factor of 1.3 and the statin/arginine treatment shows the smallest lesion surface with 8.8% lesion compared with 13.7% for the control group.

Moreover, a significant inverse correlation between total lesion surface and arginine concentration is found throughout the protocol. This correlation is also found for ornithine.

Finally, at microscopic level, it appears that for the statin/arginine group, the lesions are less thick than for the other groups.

1-22. (canceled)

23. Products containing
at least one statin, or a pharmaceutically acceptable salt thereof, and
at least one compound of the following formula (I): 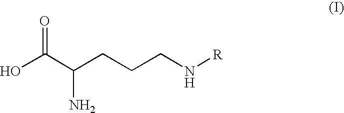
in which R represents 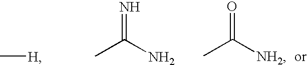
or a pharmaceutically acceptable salt thereof, as combination products for simultaneous or separate use or use spread over time within the framework of the treatment of a pathology selected from the group consisting of: atherosclerosis, hypertension, diabetes and neurodegenerative diseases.

24. Products according to claim 23, in which the statin is chosen from the group consisting of: atorvastatin, pravastatin, fluvastatin, rosuvastatin, lovastatin and simvastatin.

25. Products according to claim 23, in which the statin is atorvastatin.

26. Products according to claim 23, in which the compound of formula (I) corresponds to the L-isomer.

27. Products according to claim 23, in which R represents 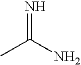
the compound of formula (I) then corresponding to arginine.

28. Products according to claim 23, suitable for the administration to an individual of a dose from 5 mg/day to 80 mg/day of statin.

29. Products according to claim 23 suitable for the administration to an individual of a dose from 1 g/day to 30 g/day of a compound of formula (I).

30. Products according to claim 23, suitable for the administration to an individual of a dose of L-Arginine of at least 10 g/day.

31. A pharmaceutical composition comprising as active ingredient:
at least one statin, or a pharmaceutically acceptable salt thereof, and
L-Arginine, or a pharmaceutically acceptable salt thereof, in combination with a pharmaceutically acceptable vehicle, said composition being suitable for the administration to an individual of a dose from 5 mg/day to 80 mg/day of statin, and of a dose from 5 g/day to 30 g/day of L-Arginine.

32. The pharmaceutical composition according to claim 31, in which the statin is chosen from the group consisting of: atorvastatin, pravastatin, fluvastatin, rosuvastatin, lovastatin and simvastatin.

33. The pharmaceutical composition according to claim 31, in which the statin is atorvastatin.

34. The pharmaceutical composition according to claim 31, suitable for the administration to an individual of a dose of L-Arginine of at least 10 g/day.

35. The pharmaceutical composition according to claim 31, suitable for administration by oral route.

36. The pharmaceutical composition according to claim 31, characterized in that it is presented in the form of a powder to be diluted, pills, sachets, tablets, capsules, or any other acceptable galenic form.

37. A method for the prevention or treatment of a pathology chosen from the group consisting of: atherosclerosis, hypertension, diabetes, and neurodegenerative diseases, said method comprising the administration of a pharmaceutically acceptable amount of:
at least one statin, or of a pharmaceutically acceptable salt thereof, in combination with
at least one compound of the following formula (I): 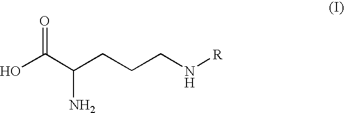
in which R represents 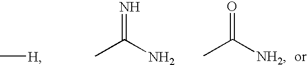
or a pharmaceutically acceptable salt thereof.

38. The method according to claim 37, in which the statin is chosen from the group consisting of: atorvastatin, pravastatin, fluvastatin, rosuvastatin, lovastatin and simvastatin.

39. The method according to claim 37, in which the statin is atorvastatin.

40. The method according to claim 37, in which the compound of formula (I) corresponds to the L-isomer.

41. The method according to claim 37, in which R represents 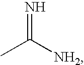
the compound of formula (I) then corresponding to arginine.

42. The method according to claim 37, wherein the statin is in a quantity suitable for the administration to an individual of a dose from 5 mg/day to 80 mg/day.

43. The method according to claim 37, wherein the compound of formula (I) is in a quantity suitable for the administration to an individual of a dose from 1 g/day to 30 g/day.

44. The method according to claim 37, wherein said compound of formula (I) is L-Arginine, said L-Arginine being in a quantity suitable for the administration to an individual of a dose of at least 10 g/day.

45. The method according to claim 37, for the prevention or treatment of Alzheimer disease.
